(19) United States
(12) Patent Application Publication
(10) Pub. No.: US 2026/0325564 A1
(43) Pub. Date: Oct. 22, 2026

(54) ERECTILE DYSFUNCTION PREVENTION AND/OR TREATMENT THEREOF

(71) Applicant: Evincis Bio Inc., Dover, DE (US)

(72) Inventors: Nikolai Sopko, Salt Lake City, UT (US); Marytheresa Ifediba, Springville, UT (US); Nicholas Baetz, Draper, UT (US)

(21) Appl. No.: 19/643,924

(22) Filed: Apr. 10, 2026

Related U.S. Application Data
(60) Provisional application No. 63/786,532, filed on Apr. 10, 2025.

(30) Foreign Application Priority Data
Apr. 18, 2025 (NL) 4000061

(51) Int. Cl. A61K 31/7105; A61K 9/00; A61K 9/127; A61K 9/51; A61K 38/19; A61P 15/10; C12N 15/11; C12N 15/88

(52) U.S. Cl. CPC A61K 31/7105; A61K 9/0034; A61K 9/127; A61K 9/5123; A61K 38/195; A61P 15/10; C12N 15/11; C12N 15/88; C12N 2310/335

(57) ABSTRACT

Compositions including a nucleotide sequence as set forth in SEQ ID NO: 1 and/or having 95% homology to SEQ ID NO:1, more preferably having 99% homology to SEQ ID NO: 1 wherein each uracil loci in SEQ ID NO: 1 has been substituted with 5-Methoxyuridine (5 MOU) and/or N1-Methylpseudouridine (N1). A method of treating and/or preventing human erectile dysfunction in a subject in need thereof by (a) contacting human penile cells with a composition having a nucleotide sequence as set forth in SEQ ID NO: 1 and/or having 95% homology to SEQ ID NO: 1, more preferably having 99% homology to SEQ ID NO: 1 wherein each uracil loci in SEQ ID NO: 1 has been substituted with 5-Methoxyuridine (5 MOU) and/or N1-Methylpseudouridine (N1).

### CROSS REFERENCE TO RELATED APPLICATIONS

This application is a nonprovisional of and claims priority to U.S. Provisional Patent Application No. 63/786,532, filed Apr. 10, 2025, and Dutch Patent Application No. 4000061, filed Apr. 18, 2025, the contents of each of which are hereby incorporated by reference into this disclosure.

### SEQUENCE LISTING

The sequence listing entitled “P103113NL Sequence Listing” in XML format, created on Apr. 16, 2025, and being 6000 bytes in size, is hereby incorporated into this disclosure.

### TECHNICAL FIELD

The present invention relates generally to the field of erectile dysfunction treatments, and more particularly, to the treatment of erectile dysfunction by the administration of modified Stromal Derived Factor-1 (SDF-1) nucleotide sequences to human penile cells and/or tissue and subsequent expression of SDF-1 peptide(s).

### BACKGROUND

Stromal derived factor 1 (SDF-1) is a small, highly conserved chemokine known for being important for bone marrow homing and B-cell proliferation and also plays a role in many diverse cellular functions, including embryogenesis, immune surveillance, inflammation response, tissue homeostasis, tissue repair, and tumor growth and metastasis.

Mutations in this gene are associated with resistance to human immunodeficiency virus type 1 infections. Multiple transcript variants encoding different isoforms have been found for this gene having various different applications.

Erectile dysfunction is a male sexual dysfunction characterized by the inability to develop or maintain an erection of the penis during sexual intercourse, which has various different causes/pathologies. The psychogenic cause, one type of cause for erectile dysfunction, is attributed to the activation of sympathetic nervous system and inhibition of non-adrenergic-non-cholinergic nerve system by psychological or mental effect. The organic erectile dysfunctions, a second type of cause of erectile dysfunction, are divided into neurogenic, vasculogenic, and endocrine erectile dysfunctions according to the cause.

Erectile dysfunction is increasingly recognized as an immune-mediated condition, with inflammation being a major contributor and perhaps originating insult to the vascular, nervous, and hormonal complications involved. This complex interplay involves cytokines, chemokines, adhesion molecules, and signaling cascades resulting in activation of cell-survival pathways which can lead to end-organ dysfunction if left unaddressed. Relevant biomarkers of inflammation include C-reactive protein, TNF-α, and the interleukins (e.g., interleukin-1 (IL-1), IL-6, IL-17), among many others. Inflammation is one of the explanatory links between erectile dysfunction and other chronic diseased states such as hyperlipidemia, diabetes mellitus, high blood pressure, smoking, metabolic syndrome, etc.

Vasculogenic erectile dysfunction is known to cause insufficient secretion of vasorelaxants (e.g., nitric oxide (NO)) from cavernous endothelial cells due to endothelial dysfunction. Chronic inflammation is a major contributor to endothelial dysfunction, shifting the endothelial cell away from homeostatic NO production and towards reactive oxygen species (ROS) production, which in turn further contributes to an inflammatory milieu. Molecules such as NADPH oxidase and uncoupled endothelial nitric oxide synthase (eNOS) generate large quantities of ROS, resulting in DNA damage, inflammatory cascades, and activation of inflammasomes both in the endothelium itself and in other cell types, such as smooth muscle. This cycle of inflammation leading to ROS, which leads to further inflammation, is a feedback loop which markedly aggravates erectile dysfunction.

Testosterone is another central inflammation-responsive molecule which contributes to nearly every system involved in generating an erection, from regulation at the genome level through androgen receptor signaling to direct relaxant effects on the vasculature at the membrane. Inflammation is well documented to decrease testosterone production, which has profound effects on endothelial NO production, the structure of penile tissue, and expression of phosphodiesterases (PDEs) such as PDE5.

Recent researchers associated with erectile dysfunction have intensely focused on organic erectile dysfunction. Phosphodiesterase-5 (PDE-5) inhibitors for oral administration including Viagra (Sildenafil's trade name) are widely utilized as first-line therapy for erectile dysfunction. Oral PDE-5 inhibitors are well known to be effective for the treatment of erectile dysfunction by potentiating the physiologic erectile response to NO and by amplifying the NO-cyclic GMP pathway through competitive inhibition of cyclic GMP degradation.

However, more than 30% of total patients with erectile dysfunction, including diabetic patients, those with spinal cord injuries and those who underwent radical prostatectomy for the treatment for prostatic cancer, etc., do not respond to oral PDE-5 inhibitors. Prostate surgery results in a temporary neuropraxia resulting in near complete temporary loss of erectile function resulting in decreased penile tissue perfusion. More seriously injured nerves undergo Wallerian degeneration resulting in loss in penile tissue innervation. Morphological tissue changes include smooth muscle apoptosis, decreased density of microvasculature, increased collagen/scar remodeling and replacement of erectile tissues seen both in humans and in animals (using the cavernous nerve crush model). Furthermore, these injuries result in Aberrant molecular signaling including increased RhoA/ROCK activation, decreased nNOS, increased free radical production, decreased growth factor expression.

Moreover, adverse events associated with PDE-5 inhibitor treatment such as headache, facial flushing, indigestion, back pain/myalgia, visual disturbance are not infrequent. In addition, these treatments also have some restrictions in their application. PDE-5 inhibitors are absolutely contraindicated for patients using nitrates and are either not recommended or to be used with caution in men with severe cardiovascular disease such as unstable angina, cardiac failure, recent myocardial infarct, poorly controlled blood pressure, in men who takes ketoconazole, itraconazole, ritonavir, alpha-adrenergic blocker, and in patients with severe hepatic insufficiency or renal insufficiency, etc. Furthermore, PDE5 inhibitors are used as on demand medicines but not effective to cure underlying disease at all, which limits the spontaneity of the sexual activity and causes higher drop-out rate of the treatment.

To address the above-mentioned problems, U.S. Pat. No. 11,583,571 disclosed various nucleotide and peptide sequences of Stromal derived factor 1 (SDF-1), with a major focus on the peptides disclosed therein, as potential candidates for treating erectile dysfunction however, the most promising peptide(s) disclosed therein exhibited only minimal therapeutic effects due to delivery and peptide stability issues. In contrast, the instant disclosure provides improved expression of SDF-1 through modified mRNA delivery wherein all uracil loci are substituted with 5-Methoxyuridine (5 MOU) and/or N1-Methylpseudouridine (N1); substantially increasing expression levels and mRNA stability and decreasing inflammatory response to the exogenous mRNA construct when compared with unmodified mRNA.

### SUMMARY

Disclosed herein is the use of nucleotides including RNA and DNA coding for and/or expressing Stromal Derived Factor-1 (SDF-1) peptide(s) to treat erectile dysfunction (ED) by delivery of the nucleotides to penile tissues, which overcome the above-mentioned problems in the prior art. In certain aspects, these nucleotides are directly contacted with penile cells and/or directly injected into penile tissues and/or topically applied to penile tissues, and/or subject to intraurethral administration. The goal of this therapy and nucleotide delivery is for the penile tissues to express SDF-1 and achieve more stability and efficacy than previous erectile dysfunction compositions. In certain aspects and as disclosed further herein, the nucleotides can be administered by themselves or in combination with a carrier including lipid nanoparticles, polymer-based, and/or conjugated proteins to facilitate delivery. In certain aspects, the nucleotide sequence(s) disclosed herein is altered to improve nucleotide delivery, stability, expression, and to reduce inflammation. In certain aspects, a further objective of the invention is delivery of an engineered/modified nucleic acid with and/or without a carrier to express SDF-1 in penile tissues to initiate tissue repair without the need of exogenous stem cells in which the objective is not just to deliver SDF-1 itself but the nucleic acid in order to induce SDF-1 expression in penile cells/tissues so that SDF-1 expression increases and subsequently treats intracellular pathways and/or pathologies associated with erectile dysfunction.

In certain aspects disclosed is a composition comprising a nucleotide sequence as set forth in SEQ ID NO: 1 and/or having 95% homology to SEQ ID NO:1, more preferably having 99% homology to SEQ ID NO: 1 wherein each uracil loci in SEQ ID NO: 1 has been substituted with 5-Methoxyuridine (5 MOU) and/or N1-Methylpseudouridine (N1). In certain aspects, the compositions disclosed herein either exclude and/or do not include stem cells therein.

In certain aspects, the composition further comprises a carrier, the carrier having SEQ ID NO: 1 therein and/or thereon and/or conjugated to SEQ ID NO: 1.

In certain aspects, the carrier is a polymeric based carrier including but not limited to polyethylene glycol-poly(disulfide amine) (PEG-PDSA) and/or the carrier is a lipid nanoparticle based carrier.

Also disclosed is a polypeptide expressed from and/or corresponding to a nucleotide sequence as set forth in SEQ ID NO: 1 and/or having 95% homology to SEQ ID NO: 1, more preferably having 99% homology to SEQ ID NO: 1.

Also disclosed is a method of treating and/or preventing erectile dysfunction in a subject in need thereof, the method comprising: (a) contacting human cells with a composition having a nucleotide sequence as set forth in SEQ ID NO: 1 and/or having 95% homology to SEQ ID NO: 1, more preferably having 99% homology to SEQ ID NO: 1 wherein each uracil loci in SEQ ID NO: 1 has been substituted with 5-Methoxyuridine (5 MOU) and/or N1-Methylpseudouridine (N1).

In certain aspects, the method further comprises (b) expressing in the human cells a polypeptide from and/or corresponding to the nucleotide sequence as set forth in SEQ ID NO: 1 and/or having 95% homology to SEQ ID NO: 1, more preferably having 99% homology to SEQ ID NO: 1.

In certain aspects, the human cells used in the method are human fibroblasts, preferably human penile fibroblasts, and/or human endothelial cells, preferably human penile endothelial cells, or combination thereof.

In certain aspects of the method, the nucleotide composition of step (a) further comprises a carrier, the carrier having SEQ ID NO: 1 therein and/or thereon and/or the carrier conjugated to SEQ ID NO: 1.

In certain aspects, the carrier of the method is a polymeric based carrier including but not limited to polyethylene glycol-poly(disulfide amine) (PEG-PDSA) and/or the carrier is a lipid nanoparticle based carrier.

In certain aspects, the method results in expression of SDF-1, wherein the expression of the polypeptide within the human penile tissues and/or human cells and/or the human penile cells is 100-fold to 5000-fold higher than expression of wild-type Stromal Derived Factor-1 (SDF-1) (SEQ ID NO: 3) in human penile tissues and/or human cells and/or human penile cells contacted with a same concentration of SEQ ID NO: 3 as the concentration of SEQ ID NO: 1.

In certain aspects, the method results in reduced expression of at least one proinflammatory cytokine, wherein expression of the at least one pro-inflammatory cytokine within the human penile tissues and/or human cells and/or the human penile cells is 4-fold to 172-fold lower than expression of the at least one pro-inflammatory cytokine in human penile tissues and/or human cells and/or human penile cells contacted with a same concentration of SEQ ID NO: 3 as the concentration of SEQ ID NO: 1.

In certain aspects, the method prevents, treats, and/or reduces erectile dysfunction pathologies, including but not limited to intracellular and/or intercellular pathways involved with/associated with erectile dysfunction and/or the symptoms thereof, in the human cells and/or human penile tissue(s).

Embodiments of the invention can include one or more or any combination of the above features and configurations.

Additional features, aspects and advantages of the invention will be set forth in the detailed description which follows, and in part will be readily apparent to those skilled in the art from that description or recognized by practicing the invention as described herein. It is to be understood that both the foregoing general description and the following detailed description present various embodiments of the invention and are intended to provide an overview or framework for understanding the nature and character of the invention as it is claimed. The accompanying figures/drawings are included to provide a further understanding of the invention and are incorporated in and constitute a part of this specification.

### BRIEF DESCRIPTION OF THE DRAWINGS

These and other features, aspects and advantages of the present invention are better understood when the following detailed description of the invention is read with reference to the accompanying drawings, in which:

FIG. 1 is a graph depicting intracellular SDF-1 expression levels/SDF-1 protein concentrations in human fibroblast cells post-transfection with various concentrations of either wild-type (“WT”) SDF-1 mRNA or genetically modified/engineered SDF-1 mRNA (SDF-1 mRNA transcripts having N1-Methylpseudouridine substituted at every uracil/uridine loci when compared with the WT SDF-1 mRNA);

FIG. 2 is a graph depicting SDF-1 expression levels/SDF-1 protein concentrations in conditioned media post-transfection of human fibroblast cells with various concentrations of either wild-type (“WT”) SDF-1 mRNA or genetically modified/engineered SDF-1 mRNA (SDF-1 mRNA transcripts having N1-Methylpseudouridine substituted at every uracil/uridine loci when compared with the WT SDF-1 mRNA);

FIG. 3 depicts green fluorescent protein levels (GFP levels) of GFP expressing mRNA combined with a commercially available lipid nanoparticle based carrier;

FIG. 4 depicts mRNA expression levels of interleukin-6 after transfection with mRNA expressing wild-type SDF-1, 5MOU SDF-1, and GFP;

FIG. 5A depicts N1-Methylpseudouridine, which is substituted for each uracil/uridine within each uracil/uridine loci as disclosed in Table 1, and FIG. 5B depicts N1-Methylpseudouridine-5′-Triphosphate; and

FIG. 6A depicts 5-Methoxyuridine, which is substituted for each uracil/uridine within each uracil/uridine loci as disclosed in Table 1 and FIG. 6B depicts 5-Methoxyuridine-5′ Triphosphate.

### DETAILED DESCRIPTION

The present invention will now be described more fully hereinafter with reference to the accompanying drawings, figures, graphs, and/or tables in which exemplary embodiments of the invention are shown. However, the invention may be embodied in many different forms and should not be construed as limited to the representative embodiments set forth herein. The exemplary embodiments are provided so that this disclosure will be both thorough and complete and will fully convey the scope of the invention and enable one of ordinary skill in the art to make, use and practice the invention. Like reference numbers refer to like elements throughout the various drawings, figures, graphs, and/or tables.

Further, the term “or” as used in this disclosure and the appended claims is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form. Throughout the specification and claims, the following terms take at least the meanings explicitly associated herein, unless the context dictates otherwise. The meanings identified below do not necessarily limit the terms but merely provide illustrative examples for the terms. The meaning of “a,” “an,” and “the” may include plural references, and the meaning of “in” may include “in,” “at,” and/or “on,” unless the context clearly indicates otherwise. The phrase “in one embodiment,” as used herein does not necessarily refer to the same embodiment, although it may.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within the ranges as if each numerical value and sub-range is explicitly recited. As an illustration, a numerical range of “about 1 to 5” should be interpreted to include not only the explicitly recited values of about 1 to about 5 but also include individual values and sub-ranges within the indicated range. Thus, included in this numerical range are individual values such as 2, 3, and 4 and sub-ranges such as from 1-3, from 2-4, and from 3-5, etc. as well as 1, 2, 3, 4, and 5, individually. The same principle applies to ranges reciting only one numerical value as a minimum or a maximum. Furthermore, such an interpretation should apply regardless of the breadth of the range or the characteristics being described.

### Compositions

Disclosed herein are nucleotide compositions including RNA that code for and/or express Stromal Derived Factor-1 (SDF-1) peptide(s). The disclosed RNA are preferably more stable than the corresponding wild type sequences thereby allowing for higher and/or more sustained expression level(s) of Stromal Derived Factor-1 (SDF-1) peptide(s), for example, within human cells including in vitro, ex vivo, in vivo, or any combination thereof. Moreover, the compositions disclosed herein may advantageously treat erectile dysfunction (ED) by more efficient delivery of the nucleotides disclosed herein to penile tissues and/or subsequent higher and/or more sustained expression level(s) of Stromal Derived Factor-1 (SDF-1) peptide(s) when compared to wild type sequences. In certain aspects, the compositions disclosed herein either exclude and/or do not include stem cells therein.

In view of the above and in further view of Table 1, the invention discloses a nucleotide sequence (DNA sequence) of wild-type SDF-1 (SEQ ID NO.: 2). Further disclosed is a corresponding mRNA sequence/mRNA transcript (SEQ ID NO.: 3) of SEQ ID NO.: 2. Moreover, further disclosed is a genetically modified/engineered SDF-1 mRNA (SEQ ID NO.: 1) when compared with the wild type sequence(s) disclosed in SEQ ID NO.: 2 and SEQ ID NO.: 3. In particular and as indicated in SEQ ID NO: 1, the bold and highlighted loci correspond to mRNA having either 5-Methoxyuridine and/or N1-Methylpseudouridine substituted at every uracil/thymine loci when compared with the corresponding wild type sequence (e.g., SEQ ID NO: 3). SEQ ID NOs.: 4 and 5 disclose the 5′ untranslated region (UTR) and 3′ untranslated region (UTR) respectively included within each of SEQ ID NOs.: 1-3.

TABLE 1
Sequences used in accordance with the invention

| | |
|---|---|
| SEQ ID NO.: 1 | AUGAACGCCAAGGUCGUGGUCGUGCUGGUCCUCGUGCUGACC |
| | GCGCUCUGCCUCAGCGACGGGAAGCCCGUCAGCCUGAGCUAC |
| | AGAUGCCCAUGCCGAUUCUUCGAAAGCCAUGUUGCCAGAGCC |
| | AACGUCAAGCAUCUCAAAAUUCUCAACACUCCAAACUGUGCC |
| | CUUCAGAUUGUAGCCCGGCUGAAGAACAACAACAGACAAGUG |
| | UGCAUUGACCCGAAGCUAAAGUGGAUUCAGGAGUACCUGGA |
| | GAAAGCUUUAAACAAGUAA |
| SEQ ID NO.: 2 | ATGAACGCCAAGGTCGTGGTCGTGCTGGTCCTCGTGCTGACCGC |
| | GCTCTGCCTCAGCGACGGGAAGCCCGTCAGCCTGAGCTACAGAT |
| | GCCCATGCCGATTCTTCGAAAGCCATGTTGCCAGAGCCAACGTC |
| | AAGCATCTCAAAATTCTCAACACTCCAAACTGTGCCCTTCAGATT |
| | GTAGCCCGGCTGAAGAACAACAACAGACAAGTGTGCATTGACCC |
| | GAAGCTAAAGTGGATTCAGGAGTACCTGGAGAAAGCTTTAAACA |
| | AGTAA |
| SEQ ID NO.: 3 | AUGAACGCCAAGGUCGUGGUCGUGCUGGUCCUCGUGCUGACCG |
| | CGCUCUGCCUCAGCGACGGGAAGCCCGUCAGCCUGAGCUACAG |
| | AUGCCCAUGCCGAUUCUUCGAAAGCCAUGUUGCCAGAGCCAAC |
| | GUCAAGCAUCUCAAAAUUCUCAACACUCCAAACUGUGCCCUUC |
| | AGAUUGUAGCCCGGCUGAAGAACAACAACAGACAAGUGUGCAU |
| | UGACCCGAAGCUAAAGUGGAUUCAGGAGUACCUGGAGAAAGC |
| | UUUAAACAAGUAA |
| SEQ ID NO.: 4 | AGGAAATAAGAGAGAAAAGAAGAGTAAGAAGAAATATAAGAGC |
| | CACC |
| SEQ ID NO.: 5 | GCTGCCTTCTGCGGGGCTTGCCTTCTGGCCATGCCCTTCTTCTCTC |
| | CCTTGCACCTGTACCTCTTGGTCTTTGAATAAAGCCTGAGTAGGA |
| | AG |

Without wishing to be bound by theory, mRNA transcripts having unmodified uracil/uridine (i.e., naturally occurring uracil/uridine) are unstable such as SEQ ID NO.: 3 (and/or mRNAs encoded for by SEQ ID NO.: 2), thus leading to a constant need to transcribe the desired mRNA to induce and/or maintain subsequent expression of the corresponding desired protein. Moreover, unmodified uracil/uridine (i.e., naturally occurring uracil/uridine) can result in increased endogenous/intracellular inflammation by inducing, for example, endogenous inflammatory pathways and/or inflammatory cascades. For example, unmodified uridine in exogenous mRNA is recognized by endosomal and cytosolic pattern recognition receptors, including Toll-like receptors 3, 7, and 8 (TLR3, TLR7, TLR8) and RIG-I/MDA-5, which activate NF-κB-mediated inflammatory signaling cascades leading to proinflammatory cytokine production including IL-6.

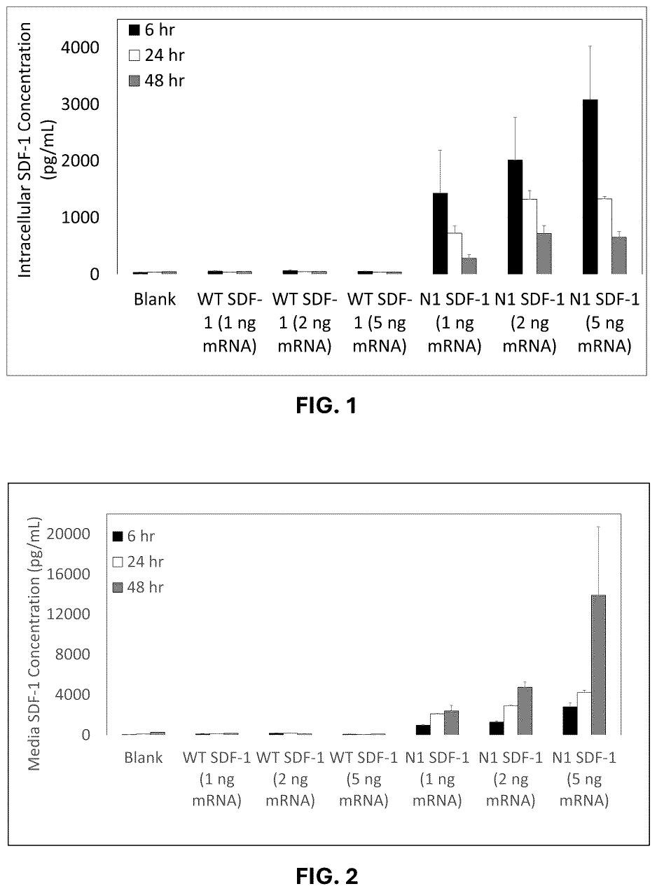

To avoid the above-mentioned disadvantages, compositions disclosed in Table 1 (SEQ ID NO.: 1) were prepared in which the mRNA sequences included modified uracil/uridine substituted at each loci where uracil (unmodified uracil/uridine) occurs in the wild type mRNA (SEQ ID NO.: 3). Without wishing to be bound by theory, the substitution of 5-Methoxyuridine at every uridine locus in SEQ ID NO.: 1 substantially evades recognition by innate immune sensors, thereby preventing or substantially reducing activation of inflammatory pathways while preserving the ability of the mRNA to be efficiently translated into SDF-1 protein. In certain aspects, the modified uracil/uridine includes 5-Methoxyuridine, N1-Methylpseudouridine, or a combination thereof in which the additional methoxy group in 5-Methoxyuridine (when compared with unmodified, naturally occurring uridine/uracil) and/or the additional methyl group in N1-Methylpseudouridine (when compared with unmodified, naturally occurring uridine/uracil) results in additional mRNA sequence stability when compared with the wild type sequence (SEQ ID NO.: 3 having unmodified uridine/uracil therein). In certain aspects, SEQ ID NO.: 1 only included a single modified uracil/uridine (e.g., either 5-Methoxyuridine, N1-Methylpseudouridine) at each loci where uracil occurs in the wild type mRNA (SEQ ID NO.: 3). In certain aspects disclosed is a composition comprising a nucleotide sequence as set forth in SEQ ID NO: 1 and/or having 95% homology to SEQ ID NO: 1, more preferably having 99% homology to SEQ ID NO: 1. As shown in FIGS. 1 and 2, the compositions disclosed herein preferably include SEQ ID NO.: 1 at concentrations ranging from 0.5 ng to 20 ng, more preferably 1 ng to 15 ng in which any concentration occurring within these ranges may be used to form any desired subrange(s), and as further shown, the subsequent expression of SDF-1 from SEQ ID NO. 1 is 10 fold to 10,000 fold higher than expression levels of SDF-1 encoded by SEQ ID NO. 3 in which any concentration occurring within these ranges may be used to form any desired subrange(s).

In certain aspects and as disclosed further herein, the nucleotide sequence(s) can be used and/or administered by themselves (e.g., SEQ ID NO.: 1 alone) or in combination with a carrier including lipid nanoparticles, polymer-based, and/or conjugated proteins to facilitate delivery. In certain preferred aspects and to further enhance SDF-1 expression levels, a carrier is present, the carrier having SEQ ID NO: 1 therein and/or thereon and/or conjugated to SEQ ID NO: 1. In certain aspects, the carrier is a polymeric based carrier including but not limited to polyethylene glycol-poly(disulfide amine) (PEG-PDSA) and/or the carrier is a lipid nanoparticle based carrier. In certain aspects, the carrier is a polymeric based carrier including but not limited to a poly(disulfide amine). In certain aspects, the poly(disulfide amine) is polyethylene glycol poly(disulfide amine) (PEG-PDSA). The poly(disulfide amine) may be poly[N,N′-bis(2-aminoethyl) cystamine-co-1,2-diaminoethane] (“poly(CBA-DAE)”), poly[N,N′-bis(2-aminoethyl) cystamine-co-1,4-diaminobutane] (“poly(CBA-DAB)”), poly(cystamine bisacrylamide-diamino hexane) (“poly(CBA-DAH)”), poly(cystaminebisacrylamide-spermine) (“poly(CBA-SP)”), poly(cystaminebisacrylamide-bis(3-aminopropyl)-1,3-propanediamine) (“poly(CBA-APPD)”), poly(cyxtaminebisacrylamide-bis(3-aminopropyl)-ethylenediamine) (“poly(CBA-APED)”), or a combination thereof. In certain aspects, the poly(disulfide amine) is represented by Formula (I), wherein n is about 1 to about 100 and R is (CH₂)ₘNH₂, wherein m is about 1 to about 18.

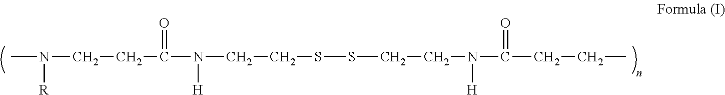

In certain aspects, the carrier is a lipid nanoparticle based carrier including but not limited to any one or a combination of the following: N-[1-(2,3-dioleyloxy)propyl]-N,N,N-trimethylamonium chloride (“DOTMA”), dioleoylphosphatidylcholine (“DOPE”), 1,2-Bis(oleoyloxy)-3-(4′-trimethylammonio) propane (“DOTAP”), 1,2-dioleoyl-3-(4′-trimethylammonio) butanoyl-sn-glycerol (“DOTB”), 1,2-dioleoyl-3-succinyl-sn-glycerol choline ester (“DOSC”), cholesteryl (4′-trimethylammonio)butanoate (“ChoTB”), cetyltrimethylammonium bromide (“CTAB”), 1,2-dioleoyl-3-dimethyl-hydroxyethyl ammonium bromide (DORI), 1,2-dioleyloxypropyl-3-dimethyl-hydroxyethyl ammonium bromide “(DORIE), 1,2-dimyristyloxypropyl-3-dimethyl-hydroxyethyl ammonium bromide (“DMRIE”), O,O′-didodecyl-N-[p(2-trimethylammonioethyloxy)benzoyl]-N,N,N-trimethylam-monium chloride, spermine conjugated to one or more lipids (for example, 5-carboxyspermylglycine dioctadecylamide (“DOGS”), N,NI,NII,NIII-tetramethyl-N,NI,NII,NIII-tetrapalmitylspermine (“TM-TPS”) and dipalmitoylphasphatidylethanolamine 5-carboxyspermylaminde (“DPPES”), lipopolylysine (polylysine conjugated to DOPE), TRIS (Tris (hydroxymethyl)aminomethane, tromethamine) conjugated fatty acids (“TFAs”) and/or peptides such as trilysyl-alanyl-TRIS mono-, di-, and tri-palmitate, (3β-[N—(N′,N′-dimethylaminoethane)-carbamoyl]cholesterol (DC-Chol), N-(α-trimethylammonioacetyl)-didodecyl-D-glutamate chloride (“TMAG”), dimethyl dioctadecylammonium bromide (DDAB), and 2,3-dioleyloxy-N-[2 (spermine-carboxamido)ethyl]-N,N-dimethyl-1-propanamin-iniumtrifluoroacetate (“DOSPA”). Suitable commercial lipid systems include: FUGENE® HD, TRANSFECTAM® (DOGS), LIPOFECTIN® (1:1 (w/w) combination of DOTMA and DOPE), LIPOFECTAMINE® (3:1 (w/w) combination of DOSPA and DOPE), LIPOFECTAMINE® 2000, LIPOFECTAMINE™ 3000, LIPOFECTAMINE® LTX, EFFECTENE®).

In certain aspects the composition(s) disclosed herein prevents, treats, and/or reduces erectile dysfunction in a human male. In certain aspects, the composition(s) prevents, treats, and/or reduces erectile dysfunction by reducing inflammation and/or pro-inflammatory cytokines, and/or innate immune activation associated with erectile dysfunction. These compositions include, but are not limited to, an injectable composition and/or a topical composition that prevents, treats, and/or reduces erectile dysfunction in a human male comprising a nucleotide sequence as set forth in SEQ ID NO: 1 and/or having 95% homology to SEQ ID NO:1, more preferably having 99% homology to SEQ ID NO: 1.

In certain aspects, the injectable and/or topical composition further comprises one of the above disclosed carrier, the carrier having SEQ ID NO: 1 (and/or having 95% homology to SEQ ID NO:1, more preferably having 99% homology to SEQ ID NO: 1) therein and/or thereon and/or conjugated to SEQ ID NO: 1 (and/or having 95% homology to SEQ ID NO:1, more preferably having 99% homology to SEQ ID NO: 1).

Method of Contacting Human Cells with the Composition(s)

As alluded to above, the compositions disclosed herein may be contacted and expressed within human cells and/or human tissues, more preferably in human penile cells and/or human penile tissues. In particular, disclosed is a method including (a) contacting human cells and/or human tissues with a composition having a nucleotide sequence as set forth in SEQ ID NO: 1 and/or having 95% homology to SEQ ID NO:1, more preferably having 99% homology to SEQ ID NO: 1. In certain aspects, the compositions disclosed herein preferably include SEQ ID NO.: 1 and/or having 95% homology to SEQ ID NO:1, more preferably having 99% homology to SEQ ID NO: 1 at concentrations ranging from 0.5 ng to 20 ng, more preferably 1 ng to 15 ng in which any concentration occurring within these ranges may be used to form any desired subrange(s). The human cells and/or human tissues may be contacted with the disclosed compositions by conventional methods including, but not limited to, transfection, lipofection, injection (local and/or systemic), topical application, and/or intraurethral administration. In certain aspects and after contacting the human cells and/or human tissues with the compositions disclosed herein, the compositions enter into the cells (e.g., cytoplasm and/or nucleus) of the human cells and/or cells comprising the human tissues.

After step (a), the method further comprises (b) expressing (e.g., in the cytoplasm and/or nucleus) in the human cells a polypeptide from and/or corresponding to the nucleotide sequence as set forth in SEQ ID NO: 1 and/or having 95% homology to SEQ ID NO: 1, more preferably having 99% homology to SEQ ID NO: 1. For example, after contacting the human cells and/or human tissues with SEQ ID NO.: 1 and/or having 95% homology to SEQ ID NO: 1, more preferably having 99% homology to SEQ ID NO: 1 at the disclosed concentrations (e.g., ranging from 0.5 ng to 20 ng, more preferably 1 ng to 15 ng) a desired amount of SDF-1 expression occurs and/or is observed within, for example, the human cells and/or human tissues in which these expression concentrations range (i.e., SDF-1 polypeptide expressed form SEQ ID NO.: 1 and/or having 95% homology to SEQ ID NO:1, more preferably having 99% homology to SEQ ID NO: 1) from 200 μg/mL to 5000 μg/mL (in which any concentration occurring within these ranges may be used to form any desired subrange(s)) during a forty-eight (48) hour time period. In certain aspects and as shown in FIGS. 1 and 2, the expression of SDF-1 from SEQ ID NO. 1 during this forty-eight (48) hour time period is advantageously 10 fold to 10,000 fold higher (in which any concentration occurring within these ranges may be used to form any desired subrange(s)) than expression levels of SDF-1 encoded by SEQ ID NO. 3 (wild type sequence). In certain aspects and in view of FIGS. 1 and 2, expression levels of SEQ. ID NO. 1 are statistically significant having P<0.001 when compared to expression levels of the wild type (SEQ ID NO. 2 and 3).

In certain aspects, the human cells are human fibroblasts and/or human endothelial cells and/or human penile fibroblasts, human neurons, human smooth muscle cells, human penile endothelial cells, or a combination thereof. The human tissues disclosed herein are preferably human penile tissue(s) including human penile tissue(s) comprised of human penile fibroblasts and/or human endothelial cells therein. In certain aspects, the compositions and methods disclosed herein either exclude and/or do not include stem cells therein.

In certain aspects, the nucleotide sequence of step (a) further comprises a carrier, the carrier having SEQ ID NO: 1 therein and/or thereon and/or conjugated to SEQ ID NO: 1. In certain aspects, the carrier is a polymeric based carrier including but not limited to a poly(disulfide amine). In certain aspects, the poly(disulfide amine) is polyethylene glycol poly(disulfide amine) (PEG-PDSA). The poly(disulfide amine) may be poly[N,N′-bis(2-aminoethyl) cystamine-co-1,2-diaminoethane] (“poly(CBA-DAE)”), poly[N,N′-bis(2-aminoethyl) cystamine-co-1,4-diaminobutane] (“poly(CBA-DAB)”), poly(cystamine bisacrylamide-diamino hexane) (“poly(CBA-DAH)”), poly(cystaminebisacrylamide-spermine) (“poly(CBA-SP)”), poly(cystaminebisacrylamide-bis(3-aminopropyl)-1,3-propanediamine) (“poly(CBA-APPD)”), poly(cyxtaminebisacrylamide-bis(3-aminopropyl)-ethylenediamine) (“poly(CBA-APED)”), or a combination thereof. In certain aspects, the poly(disulfide amine) is represented by Formula (I), wherein n is about 1 to about 100 and R is (CH₂)ₘNH₂, wherein m is about 1 to about 18.

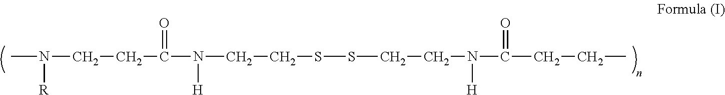

In certain aspects, the carrier is a lipid nanoparticle based carrier including but not limited to any one or a combination of the following: N-[1-(2,3-dioleyloxy)propyl]-N,N,N-trimethylamonium chloride (“DOTMA”), dioleoylphosphatidylcholine (“DOPE”), 1,2-Bis(oleoyloxy)-3-(4′-trimethylammonio) propane (“DOTAP”), 1,2-dioleoyl-3-(4′-trimethylammonio) butanoyl-sn-glycerol (“DOTB”), 1,2-dioleoyl-3-succinyl-sn-glycerol choline ester (“DOSC”), cholesteryl (4′-trimethylammonio)butanoate (“ChoTB”), cetyltrimethylammonium bromide (“CTAB”), 1,2-dioleoyl-3-dimethyl-hydroxyethyl ammonium bromide (DORI), 1,2-dioleyloxypropyl-3-dimethyl-hydroxyethyl ammonium bromide “(DORIE), 1,2-dimyristyloxypropyl-3-dimethyl-hydroxyethyl ammonium bromide (“DMRIE”), O,O′-didodecyl-N-[p(2-trimethylammonioethyloxy)benzoyl]-N,N,N-trimethylam-monium chloride, spermine conjugated to one or more lipids (for example, 5-carboxyspermylglycine dioctadecylamide (“DOGS”), N,NI,NII,NIII-tetramethyl-N,NI,NII,NIII-tetrapalmitylspermine (“TM-TPS”) and dipalmitoylphasphatidylethanolamine 5-carboxyspermylaminde (“DPPES”), lipopolylysine (polylysine conjugated to DOPE), TRIS (Tris (hydroxymethyl)aminomethane, tromethamine) conjugated fatty acids (“TFAs”) and/or peptides such as trilysyl-alanyl-TRIS mono-, di-, and tri-palmitate, (3β-[N—(N′,N′-dimethylaminoethane)-carbamoyl]cholesterol (DC-Chol), N-(α-trimethylammonioacetyl)-didodecyl-D-glutamate chloride (“TMAG”), dimethyl dioctadecylammonium bromide (DDAB), and 2,3-dioleyloxy-N-[2 (spermine-carboxamido)ethyl]-N,N-dimethyl-1-propanamin-iniumtrifluoroacetate (“DOSPA”). Suitable commercial lipid systems include: FUGENE® HD, TRANSFECTAM® (DOGS), LIPOFECTIN® (1:1 (w/w) combination of DOTMA and DOPE), LIPOFECTAMINE® (3:1 (w/w) combination of DOSPA and DOPE), LIPOFECTAMINE® 2000, LIPOFECTAMINE™ 3000, LIPOFECTAMINE® LTX, EFFECTENE®).

### Treatments

In certain aspects, the disclosed compositions and methods prevent, treat, and/or reduce erectile dysfunction pathologies in mammals and more particularly humans, including but not limited to intracellular and/or intercellular pathways involved with/associated with erectile dysfunction and/or the symptoms thereof, in the human cells and/or human penile tissue(s). This method includes treating and/or preventing human erectile dysfunction in a subject in need thereof including: (a) contacting human penile tissue(s) and/or human penile cells with a composition having a nucleotide sequence as set forth in SEQ ID NO: 1 and/or having 95% homology to SEQ ID NO:1, more preferably having 99% homology to SEQ ID NO: 1. In certain aspects, the compositions disclosed herein preferably include SEQ ID NO.: 1 and/or having 95% homology to SEQ ID NO: 1, more preferably having 99% homology to SEQ ID NO: 1 at concentrations ranging from 0.5 ng to 100,000 ng, more preferably 1 ng to 100,000 ng, even more preferably 500 ng to 100,000 ng in which any concentration occurring within these ranges may be used to form any desired subrange(s). The human penile cells and/or human penile tissues may be contacted with the disclosed compositions by conventional methods including, but not limited to, transfection, lipofection, injection (local and/or systemically), topical application, and/or intraurethral administration. In certain aspects and after contacting the human penile cells and/or human penile tissues with the compositions disclosed herein, the compositions enter into the cells (e.g., cytoplasm and/or nucleus) of the human penile cells and/or cells comprising the human penile tissues.

In certain aspects and after step (a), this method further comprises: (b) expressing in the human penile tissue(s) and/or human penile cells (e.g., in the cytoplasm and/or nucleus) a polypeptide from and/or corresponding to the nucleotide sequence as set forth in SEQ ID NO: 1 and/or having 95% homology to SEQ ID NO:1, more preferably having 99% homology to SEQ ID NO: 1 thereby preventing, treating, and/or reducing erectile dysfunction pathologies in mammals and more particularly humans, including but not limited to intracellular and/or intercellular pathways involved with/associated with erectile dysfunction and/or the symptoms thereof, in the human cells and/or human penile tissue(s). In step (b) and after contacting the human penile cells and/or human penile tissues with SEQ ID NO.: 1 and/or having 95% homology to SEQ ID NO: 1, more preferably having 99% homology to SEQ ID NO: 1 at the disclosed concentrations (e.g., ranging from 0.5 ng to 100,000 ng, more preferably 1 ng to 100,000 ng, even more preferably 500 ng to 100,000 ng) a desired amount of SDF-1 expression occurs and/or is observed within, for example, the human penile cells and/or human penile tissues in which these expression concentrations range (i.e., SDF-1 polypeptide expressed from SEQ ID NO.: 1 and/or having 95% homology to SEQ ID NO: 1, more preferably having 99% homology to SEQ ID NO: 1) from 200 μg/mL to 20,000 μg/mL (in which any concentration occurring within these ranges may be used to form any desired subrange(s)) during a forty-eight (48) hour time period. In certain aspects and as shown in FIGS. 1 and 2, the expression of SDF-1 from SEQ ID NO. 1 during this forty-eight (48) hour time period is advantageously 10 fold to 50,000 fold higher (in which any concentration occurring within these ranges may be used to form any desired subrange(s)) than expression levels of SDF-1 encoded by SEQ ID NO. 3 (wild type sequence). In certain aspects and in view of FIGS. 1 and 2, expression levels of SEQ. ID NO. 1 are statistically significant having P<0.001 when compared to expression levels of the wild type (SEQ ID NO. 2 and 3). In certain aspects, the compositions and methods disclosed herein either exclude and/or do not include stem cells therein.

In certain aspects of this method, the human penile tissue(s) and/or human penile cells are comprised of human penile fibroblasts, human neurons, human smooth muscle cells, human penile endothelial cells, or a combination thereof.

In certain aspects, the nucleotide sequence of step (a) further comprises a carrier, the carrier having SEQ ID NO: 1 therein and/or thereon and/or conjugated to SEQ ID NO: 1. In certain aspects, the carrier is a polymeric based carrier including but not limited to a poly(disulfide amine). In certain aspects, the poly(disulfide amine) is polyethylene glycol poly(disulfide amine) (PEG-PDSA). The poly(disulfide amine) may be poly[N,N′-bis(2-aminoethyl) cystamine-co-1,2-diaminoethane] (“poly(CBA-DAE)”), poly[N,N′-bis(2-aminoethyl) cystamine-co-1,4-diaminobutane] (“poly(CBA-DAB)”), poly(cystamine bisacrylamide-diamino hexane) (“poly(CBA-DAH)”), poly(cystaminebisacrylamide-spermine) (“poly(CBA-SP)”), poly(cystaminebisacrylamide-bis(3-aminopropyl)-1,3-propanediamine) (“poly(CBA-APPD)”), poly(cyxtaminebisacrylamide-bis(3-aminopropyl)-ethylenediamine) (“poly(CBA-APED)”), or a combination thereof. In certain aspects, the poly(disulfide amine) is represented by Formula (I), wherein n is about 1 to about 100 and R is (CH₂)ₘNH₂, wherein m is about 1 to about 18.

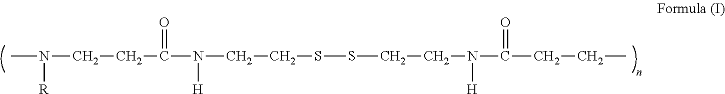

In certain aspects, the carrier is a lipid nanoparticle based carrier including but not limited to any one or a combination of the following: N-[1-(2,3-dioleyloxy)propyl]-N,N,N-trimethylamonium chloride (“DOTMA”), dioleoylphosphatidylcholine (“DOPE”), 1,2-Bis(oleoyloxy)-3-(4′-trimethylammonio) propane (“DOTAP”), 1,2-dioleoyl-3-(4′-trimethylammonio) butanoyl-sn-glycerol (“DOTB”), 1,2-dioleoyl-3-succinyl-sn-glycerol choline ester (“DOSC”), cholesteryl (4′-trimethylammonio)butanoate (“ChoTB”), cetyltrimethylammonium bromide (“CTAB”), 1,2-dioleoyl-3-dimethyl-hydroxyethyl ammonium bromide (DORI), 1,2-dioleyloxypropyl-3-dimethyl-hydroxyethyl ammonium bromide “(DORIE), 1,2-dimyristyloxypropyl-3-dimethyl-hydroxyethyl ammonium bromide (“DMRIE”), O,O′-didodecyl-N-[p(2-trimethylammonioethyloxy)benzoyl]-N,N,N-trimethylam-monium chloride, spermine conjugated to one or more lipids (for example, 5-carboxyspermylglycine dioctadecylamide (“DOGS”), N,NI,NII,NIII-tetramethyl-N,NI,NII,NIII-tetrapalmitylspermine (“TM-TPS”) and dipalmitoylphasphatidylethanolamine 5-carboxyspermylaminde (“DPPES”), lipopolylysine (polylysine conjugated to DOPE), TRIS (Tris (hydroxymethyl)aminomethane, tromethamine) conjugated fatty acids (“TFAs”) and/or peptides such as trilysyl-alanyl-TRIS mono-, di-, and tri-palmitate, (3β-[N—(N′,N′-dimethylaminoethane)-carbamoyl]cholesterol (DC-Chol), N-(α-trimethylammonioacetyl)-didodecyl-D-glutamate chloride (“TMAG”), dimethyl dioctadecylammonium bromide (DDAB), and 2,3-dioleyloxy-N-[2 (spermine-carboxamido)ethyl]-N,N-dimethyl-1-propanamin-iniumtrifluoroacetate (“DOSPA”). Suitable commercial lipid systems include: FUGENE® HD, TRANSFECTAM® (DOGS), LIPOFECTIN® (1:1 (w/w) combination of DOTMA and DOPE), LIPOFECTAMINE® (3:1 (w/w) combination of DOSPA and DOPE), LIPOFECTAMINE® 2000, LIPOFECTAMINE® 3000, LIPOFECTAMINE® LTX, EFFECTENE®).

In certain aspects, the disclosed compositions and methods prevent, treat, and/or reduce erectile dysfunction pathologies in mammals and more particularly humans, including but not limited to reducing inflammation and/or pro-inflammatory cytokines, and/or innate immune activation associated with erectile dysfunction. This method includes treating and/or preventing human erectile dysfunction in a subject in need thereof by reducing inflammation and/or pro-inflammatory cytokines, and/or innate immune activation associated with erectile dysfunction including: (a) contacting human penile tissue(s) and/or human penile cells with a composition having a nucleotide sequence as set forth in SEQ ID NO: 1 and/or having 95% homology to SEQ ID NO: 1, more preferably having 99% homology to SEQ ID NO: 1. In certain aspects, the compositions disclosed herein preferably include SEQ ID NO.: 1 and/or a sequence having 95% homology to SEQ ID NO:1, more preferably having 99% homology to SEQ ID NO: 1 at concentrations ranging from ranging from 0.5 ng to 100,000 ng, more preferably 1 ng to 100,000 ng, even more preferably 500 ng to 100,000 ng, in which any concentration occurring within these ranges may be used to form any desired subrange(s). The human penile cells and/or human penile tissues may be contacted with the disclosed compositions by conventional methods including, but not limited to, transfection, lipofection, injection (local and/or systemically), topical application, and/or intraurethral administration. In certain aspects and after contacting the human penile cells and/or human penile tissues with the compositions disclosed herein, the compositions enter into the cells (e.g., cytoplasm and/or nucleus) of the human penile cells and/or cells comprising the human penile tissues.

In certain aspects and after step (a), this method further comprises: (b) expressing in the human penile tissue(s) and/or human penile cells (e.g., in the cytoplasm and/or nucleus) a polypeptide from and/or corresponding to the nucleotide sequence as set forth in SEQ ID NO: 1 and/or a sequence having 95% homology to SEQ ID NO:1, more preferably having 99% homology to SEQ ID NO: 1 thereby preventing, treating, and/or reducing erectile dysfunction pathologies in mammals and more particularly humans, including but not limited to intracellular and/or intercellular pathways involved with/associated with erectile dysfunction and/or the symptoms thereof including but not limited to reducing inflammation and/or pro-inflammatory cytokines, and/or innate immune activation associated with erectile dysfunction, in the human cells and/or human penile tissue(s). In step (b) and after contacting the human penile cells and/or human penile tissues with SEQ ID NO.: 1 and/or a sequence having 95% homology to SEQ ID NO: 1, more preferably having 99% homology to SEQ ID NO: 1 at the disclosed concentrations (e.g., ranging from ranging from 0.5 ng to 100,000 ng, more preferably 1 ng to 100,000 ng, even more preferably 500 ng to 100,000 ng) a desired amount of reduction of pro-inflammatory cytokines including but not limited to IL-6 occurs and/or is observed within, for example, the human penile cells and/or human penile tissues. In certain aspects and as shown in FIG. 4, the reduction of IL-6 from SDF-1 from SEQ ID NO. 1 is advantageously 3.9 fold to 172.2 fold lower (in which any concentration occurring within these ranges may be used to form any desired subrange(s)) than expression levels of SDF-1 encoded by SEQ ID NO. 3 (wild type sequence). In certain aspects, the compositions and methods disclosed herein either exclude and/or do not include stem cells therein.

In certain aspects of this method, the human penile tissue(s) and/or human penile cells are comprised of human penile fibroblasts, human neurons, human smooth muscle cells, human penile endothelial cells, or a combination thereof.

In certain aspects, the composition of step (a) further comprises a carrier, the carrier having SEQ ID NO: 1 therein and/or thereon and/or the carrier conjugated to SEQ ID NO: 1. In certain aspects, the carrier is a polymeric based carrier including but not limited to a poly(disulfide amine). In certain aspects, the poly(disulfide amine) is polyethylene glycol poly(disulfide amine) (PEG-PDSA). The poly(disulfide amine) may be poly[N,N′-bis(2-aminoethyl) cystamine-co-1,2-diaminoethane] (“poly(CBA-DAE)”), poly[N,N′-bis(2-aminoethyl) cystamine-co-1,4-diaminobutane] (“poly(CBA-DAB)”), poly(cystamine bisacrylamide-diamino hexane) (“poly(CBA-DAH)”), poly(cystaminebisacrylamide-spermine) (“poly(CBA-SP)”), poly(cystaminebisacrylamide-bis(3-aminopropyl)-1,3-propanediamine) (“poly(CBA-APPD)”), poly(cyxtaminebisacrylamide-bis(3-aminopropyl)-ethylenediamine) (“poly(CBA-APED)”), or a combination thereof. In certain aspects, the poly(disulfide amine) is represented by Formula (I), wherein n is about 1 to about 100 and R is (CH₂)ₘNH₂, wherein m is about 1 to about 18.

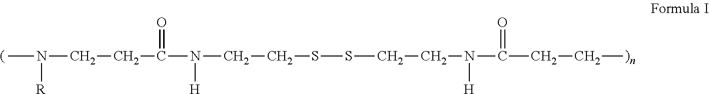

In certain aspects, the carrier is a lipid nanoparticle based carrier including but not limited to any one or a combination of the following: N-[1-(2,3-dioleyloxy)propyl]-N,N,N-trimethylamonium chloride (“DOTMA”), dioleoylphosphatidylcholine (“DOPE”), 1,2-Bis(oleoyloxy)-3-(4′-trimethylammonio) propane (“DOTAP”), 1,2-dioleoyl-3-(4′-trimethylammonio) butanoyl-sn-glycerol (“DOTB”), 1,2-dioleoyl-3-succinyl-sn-glycerol choline ester (“DOSC”), cholesteryl (4′-trimethylammonio)butanoate (“ChoTB”), cetyltrimethylammonium bromide (“CTAB”), 1,2-dioleoyl-3-dimethyl-hydroxyethyl ammonium bromide (DORI), 1,2-dioleyloxypropyl-3-dimethyl-hydroxyethyl ammonium bromide “(DORIE), 1,2-dimyristyloxypropyl-3-dimethyl-hydroxyethyl ammonium bromide (“DMRIE”), O,O′-didodecyl-N-[p(2-trimethylammonioethyloxy)benzoyl]-N,N,N-trimethylam-monium chloride, spermine conjugated to one or more lipids (for example, 5-carboxyspermylglycine dioctadecylamide (“DOGS”), N,NI,NII,NIII-tetramethyl-N,NI,NII,NIII-tetrapalmitylspermine (“TM-TPS”) and dipalmitoylphasphatidylethanolamine 5-carboxyspermylaminde (“DPPES”), lipopolylysine (polylysine conjugated to DOPE), TRIS (Tris (hydroxymethyl)aminomethane, tromethamine) conjugated fatty acids (“TFAs”) and/or peptides such as trilysyl-alanyl-TRIS mono-, di-, and tri-palmitate, (3β-[N—(N′,N′-dimethylaminoethane)-carbamoyl]cholesterol (DC-Chol), N-(α-trimethylammonioacetyl)-didodecyl-D-glutamate chloride (“TMAG”), dimethyl dioctadecylammonium bromide (DDAB), and 2,3-dioleyloxy-N-[2 (spermine-carboxamido)ethyl]-N,N-dimethyl-1-propanamin-iniumtrifluoroacetate (“DOSPA”). Suitable commercial lipid systems include: FUGENE® HD, TRANSFECTAM® (DOGS), LIPOFECTIN® (1:1 (w/w) combination of DOTMA and DOPE), LIPOFECTAMINE® (3:1 (w/w) combination of DOSPA and DOPE), LIPOFECTAMINE® 2000, LIPOFECTAMINE® 3000, LIPOFECTAMINE® LTX, EFFECTENE®).

The foregoing description provides embodiments of the invention by way of example only. It is envisioned that other embodiments may perform similar functions and/or achieve similar results. Any and all such equivalent embodiments and examples are within the scope of the present invention and are intended to be covered by the appended claims.

### WORKING EXAMPLES

### Example 1

Cell Culture and Transfection Protocol for FIGS. 1 and 2 (transfecting Human Fibroblasts (ATCC Cell #PCS-201-012) with LIPOFECTAMINE® 3000 (LIPOFECTAMINE®3000: https://www.thermofisher.com/order/catalog/product/L3000001 (accessed Apr. 8, 2025))
1. Plate cells, with a 5×10⁴ cells/500 μL culture medium (DMEM containing 10% FBS and 1% antibiotics) on 24 well plates.
2. Incubate the cells at 37° C. in 5% CO₂ for 24 hours. Cells should be 70-90% confluent.
3. Wash the cells with PBS using 500 μL
4. Replace the cultured media with serum-free DMEM media and incubated the cells for another 30 min. Opti-MEM™ can also be used.
5. Dilute LIPOFECTAMINE® 3000 reagent in Opti-MEM medium into two tubes by adding 25 μL of Opti-MEM™ in each tube and adding 0.75 μL to one tube and 1.5 μL to the other tube. This is the diluted LIPOFECTAMINE® 3000 reagent.
6. Prepare master mix of mRNA by diluting 1 μg of mRNA in 50 μL of Opti-MEM™ medium and then add 2 μL of P3000 reagent and mix well.
7. Add 25 μL of master mix with mRNA to each tube of diluted LIPOFECTAMINE® 3000 reagent.
8. Incubate for 10-15 minutes at room temperature.
9. Add 50 μL of mRNA-lipid complex to cells and incubate at 37° C. Collect cells at 6, 24, and 48 hours after transfection.

FIG. 1 discloses the intracellular concentrations of the SDF-1 protein. The intracellular concentrations were measured in vitro in human fibroblast cells transfected with either WT SDF-1 mRNA (SEQ ID NO 2 shown in Table 1) or engineered SDF-1 mRNA (i.e., “N1 SDF-1” SEQ ID NO 1 shown in Table 1). A commercially available lipid nanoparticle-based carrier (LIPOFECTAMINE® 3000) was utilized. Cells were collected 6 hr, 24 hr, and 48 hr after transfection. The SDF-1 protein levels within the cells were measured with ELISA. The intracellular concentration of the SDF-1 protein with 1 ng, 2 ng, and 5 ng of WT SDF-1 mRNA was around 0 pg/mL after 6 hr, 24 hr, and 48 hr, similar to the blank of the experiment. Thus, the WT SDF-1 mRNA did not exhibit a significant intracellular concentration of the SDF-1 protein. Conversely, the intracellular concentration of the SDF-1 protein with 1 ng of N1 SDF-1 mRNA was about 1500 pg/mL after 6 hr. The concentration decreased to about 800 pg/mL after 24 hr and 300 pg/mL after 48 hr. The intracellular concentration of the SDF-1 protein with 2 ng of N1 SDF-1 mRNA was about 2000 pg/mL after 6 hr. The concentration decreased to about 1400 pg/mL after 24 hr and 800 pg/mL after 48 hr. The intracellular concentration of the SDF-1 protein with 5 ng of N1 SDF-1 mRNA was about 3100 pg/mL after 6 hr. The concentration decreased to about 1400 pg/mL after 24 hr and 700 pg/mL after 48 hr. Overall, the N1 SDF-1 mRNA at 2 ng had roughly the same intracellular concentration of the SDF-1 protein than the N1 SDF-1 mRNA at 5 ng. Furthermore, the N1 SDF-1 mRNA at 2 ng had a higher intracellular concentration of the SDF-1 protein than the N1 SDF-1 mRNA at 1 ng and 5 ng.

Human fibroblast cells transfected with WT SDF-1 mRNA demonstrated minimal protein expression. However, human fibroblast cells transfected with the engineered N1 SDF-1 mRNA exhibited significant protein expression. Thus, engineered N1 SDF-1 mRNA is necessary for increased protein expression.

FIG. 2 discloses the concentration of the SDF-1 protein in conditioned media. The conditioned media concentrations were measured in vitro in human fibroblast cells transfected with either WT SDF-1 mRNA or engineered SDF-1 mRNA. A commercially available lipid nanoparticle-based carrier was utilized. Cells were collected 6 hr, 24 hr, and 48 hr after transfection. The SDF-1 protein levels in the media were measured with ELISA. The concentration of the SDF-1 protein in conditioned media with 1 ng, 2 ng, and 5 ng of WT SDF-1 mRNA was around 0 pg/mL after 6 hr, 24 hr, and 48 hr, similar to the blank of the experiment. Thus, the WT SDF-1 mRNA did not exhibit a significant concentration of the SDF-1 protein in the conditioned media. Conversely, the concentration of the SDF-1 protein in the conditioned media with 1 ng of N1 SDF-1 mRNA was about 1000 pg/mL after 6 hr. The concentration increased to about 2000 pg/mL after 24 hr and 2500 pg/mL after 48 hr. The concentration of the SDF-1 protein in the conditioned media with 2 ng of N1 SDF-1 mRNA was about 1200 pg/mL after 6 hr. The concentration increased to about 3000 pg/mL after 24 hr and 5000 pg/mL after 48 hr. The concentration of the SDF-1 protein in the conditioned media with 5 ng of N1 SDF-1 mRNA was about 3000 pg/mL after 6 hr. The concentration increased to about 4200 pg/mL after 24 hr and 14000 pg/mL after 48 hr. Overall, the N1 SDF-1 mRNA at 5 ng had higher concentrations of the SDF-1 protein in the conditioned media than the N1 SDF-1 mRNA at 1 ng and 2 ng.

Human fibroblast cells transfected with WT SDF-1 mRNA exhibited minimal SDF-1 protein concentrations in the conditioned media. However, human fibroblast cells transfected with the engineered N1 SDF-1 mRNA exhibited significant SDF-1 protein concentrations in the conditioned media. Thus, SDF-1 mRNA modification is necessary for increased release of the SDF-1 protein by the cells.

### Example 2

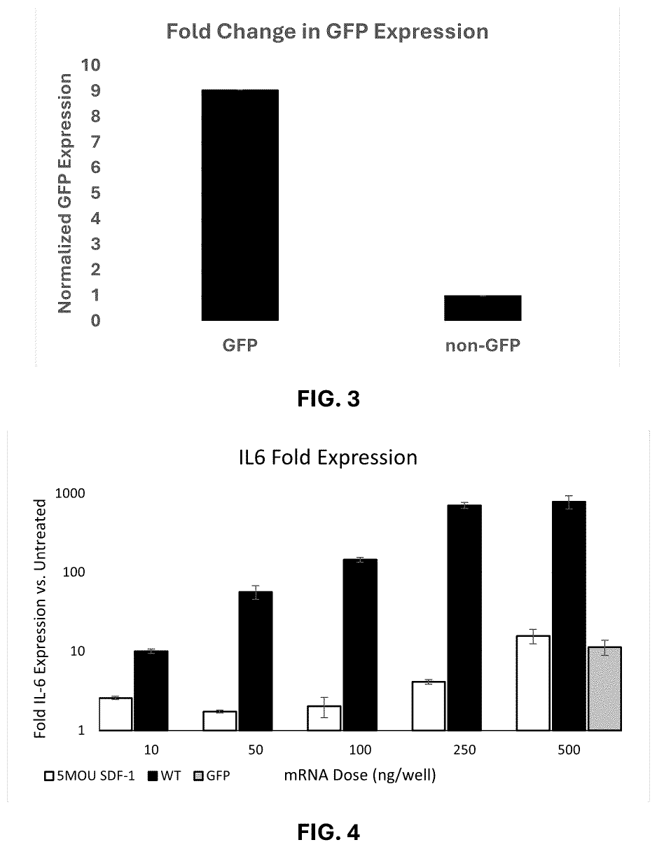

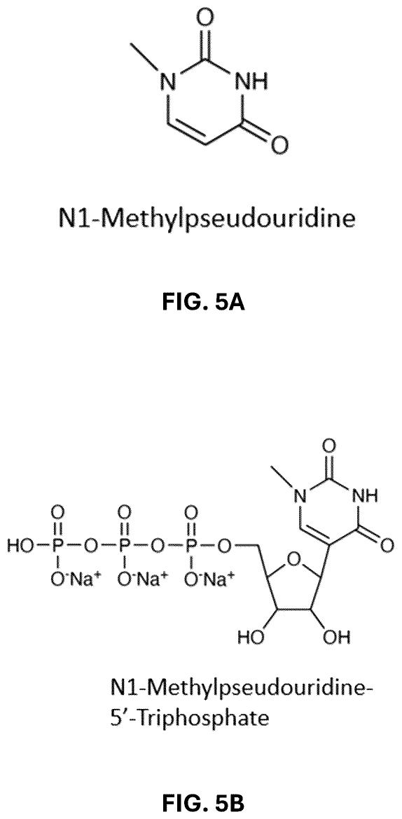

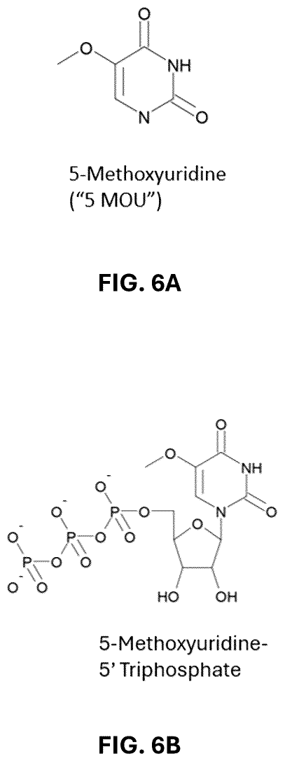

FIG. 3 demonstrates proof of concept that protein expression after injection into penile tissues occurs after administration of mRNA. In particular, FIG. 3 discloses the concentration in vivo of wild type green fluorescent protein (GFP) in rat penile tissue expressed post-injection. 5 μg of GFP-mRNA was combined with a commercially available lipid nanoparticle and injected into rat penile tissues. The tissues were collected 48 hr after injection. The GFP levels were measured with ELISA. The GFP levels were compared to non-GFP levels. The normalized GFP expression of the GFP was 9, whereas the normalized GFP expression of the non-GFP was 1. Thus, mRNA can be successfully delivered to penile tissues and result in targeted protein expression.

### Example 3

To assess the inflammatory/immunogenic potential of the nucleotide compositions disclosed herein, experiments were conducted comparing the innate immune response elicited by wild-type (WT) SDF-1 mRNA (SEQ ID NO.: 3) to genetically modified/engineered SDF-1 mRNA having 5-Methoxyuridine (5MOU) substituted at every uracil/uridine loci (SEQ ID NO.: 1) in human cells to determine whether the complete substitution of naturally occurring uridine with 5-Methoxyuridine confers advantages beyond enhanced mRNA stability and protein expression, namely a significant reduction in the inflammatory response induced by the mRNA compositions upon cellular contact. Interleukin-6 (IL-6) was selected as the readout for innate immune activation because it is a well-established downstream effector of Toll-like receptor (TLR) and RIG-I/MDA-5 pattern recognition pathways that are activated by exogenous unmodified mRNA. Cell Culture and Transfection

Protocol for FIG. 4 (transfecting Human Fibroblasts (ATCC Cell #PCS-201-012) with Lipofectamine MessengerMAX (Thermo Fisher Scientific: https://www.thermofisher.com/order/catalog/product/LMRNA001 (accessed Mar. 31, 2026) according to manufacturer instructions).
1. Plate cells at a seeding density of 2.0×10⁴ per well of a 96 well flat bottom tissue culture plate.
2. Incubate the cells at 37° C. in 5% CO₂ for 24 hours. Cells should be 70-80% confluent at the time of transfection.
3. 0.3 μL Lipofectamine MessengerMAX was mixed with 5 μL of Opti-MEM I Reduced Serum Medium (OptiMEM) per well to be transfected and incubated for 10 minutes at room temperature.
4. In a separate tube, the indicated concentration of WT SDF-1, 5MOU SDF-1, and GFP mRNA was diluted with 5 μL OptiMEM per well to be transfected.
5. After 10 minutes to separate incubation, the Lipofectamine MessengerMAX and mRNA tubes were combined, gently mixed, and incubated for 5 minutes at room temperature.
6. The resulting 10 μL complex per well was added dropwise to each well containing 90 μL antibiotic-free complete DMEM, for a final concentration of 100 uL per well.

Cells were harvested 16 hours post-transfection. Culture medium was aspirated and total RNA was extracted using the PureLink RNA Mini Kit (Thermo Fisher Scientific: https://www.thermofisher.com/order/catalog/product/12183018A (accessed Mar. 31, 2026)) according to the manufacturer's standard protocol. RNA concentration and purity were assessed by spectrophotometry (Nanodrop). All samples were normalized to an equivalent RNA input for reverse transcription.

Complementary DNA (cDNA) synthesis and quantitative PCR (qPCR) were performed simultaneously using the TaqPath 1-Step RT-qPCR Master Mix (Thermo Fisher Scientific: https://www.thermofisher.com/order/catalog/product/A15300 (accessed Mar. 31, 2026)) in a single-step reaction, according to the manufacturer's instructions. IL-6 mRNA abundance was quantified using a commercially validated TaqMan Gene Expression Assay targeting human IL-6 on a QuantStudio 12K Flex Real-Time PCR System (Thermo Fisher Scientific; Instrument ID: QS12K-2, Serial No. 285881205). Gene expression data were analyzed using the comparative CT method, with results expressed as fold change relative to untreated/mock control cells. Statistical significance was determined by independent samples two-tailed Student's t-test. A Bonferroni correction was applied for 14 comparisons, yielding a corrected significance threshold of P<0.0036. Three biological replicates (n=3) were performed for each condition.

The following control conditions were included: (i) mock/untreated cells (no mRNA, no transfection reagent); (ii) vehicle control (Lipofectamine MessengerMAX™ reagent only, no mRNA); (iii) lipopolysaccharide (LPS) positive control for NF-κB-mediated inflammatory pathway activation; and (iv) wild-type (unmodified) green fluorescent protein (GFP) mRNA at 500 ng/well as a sequence-independent unmodified mRNA control to confirm that the observed inflammatory response is attributable to unmodified uridine content rather than the SDF-1-specific mRNA sequence. Each mRNA construct (WT SDF-1, 5MOU SDF-1, and GFP) was tested at five concentrations: 10, 50, 100, 250, and 500 ng per well, in biological triplicate.

FIG. 4 depicts fold-change of mRNA expression levels of interleukin-6, a major inflammatory cytokine produced in response to cellular stress, in cells transfected with the indicated amounts of mRNA. As shown in the image, delivery of uracil-modified SDF-1 results in a reduced inflammatory response compared to wild type SDF-1 mRNA delivery.

As shown in Table 2 below, transfection with wild-type SDF-1 mRNA (SEQ ID NO: 3) induced a strong, dose-dependent inflammatory response as measured by IL-6 expression.

TABLE 2
Wild-type SD-1 vs. Mock

| | ng | | | |
|---|---|---|---|---|
| | mRNA/ | Mean Ct | ΔCt vs. | Fold vs. |
| Sample | well | (IL-6) | Mock | Mock |
| WT SDF-1 | 500 | 22.82 ± 0.48 | 9.62 | 785.7-fold (±150.3 |
| (500 ng) | | | | SEM; P = 0.00001) |
| WT SDF-1 | 250 | 22.97 ± 0.21 | 9.47 | 709.5-fold (±60.3 |
| (250 ng) | | | | SEM; P < 0.0001) |
| WT SDF-1 | 100 | 25.26 ± 0.17 | 7.18 | 144.8-fold (±9.9 |
| (100 ng) | | | | SEM; P < 0.0001) |
| WT SDF-1 | 50 | 26.62 ± 0.49 | 5.82 | 56.5-fold (±11.0 |
| (50 ng) | | | | SEM; P = 0.00005) |
| WT SDF-1 | 10 | 29.11 ± 0.16 | 3.33 | (±0.66 SEM; P < |
| (10 ng) | | | | 0.0001) |

Conversely, transfection with 5MOU-modified SDF-1 mRNA (SEQ ID NO: 1) produced a significantly lower inflammatory response across all concentrations tested. (Table 3). While the 5MOU-modified mRNA did show a modest dose-dependent increase in IL-6 induction at the two highest concentrations (250 and 500 ng/well), this response was dramatically reduced compared to the corresponding WT mRNA concentrations. Notably, at concentrations of 10 to 100 ng/well, 5MOU-modified SDF-1 mRNA induced IL-6 levels that were near baseline (approximately 1.7- to 2.6-fold), indicating near-complete evasion of innate immune detection at these therapeutically relevant concentrations.

TABLE 3
5MOU-modified SDF-1 mRNA versus Mock

| | ng | | | |
|---|---|---|---|---|
| | mRNA/ | Mean Ct | ΔCt vs. | Fold vs. |
| Sample | well | (IL-6) | Mock | Mock |
| 5MOU SDF-1 | 500 | 28.47 ± 0.52 | 3.97 | 15.6-fold relative |
| (500 ng) | | | | to mock (±3.2 |
| | | | | SEM) |
| 5MOU SDF-1 | 250 | 30.40 ± 0.17 | 2.04 | 4.1-fold relative to |
| (250 ng) | | | | mock (±0.28 SEM) |
| 5MOU SDF-1 | 100 | 31.42 ± 0.71 | 1.02 | 2.0-fold relative to |
| (100 ng) | | | | mock (±0.58 SEM; |
| | | | | P = 0.08) |
| 5MOU SDF-1 | 50 | 31.64 ± 0.10 | 0.80 | 1.7-fold relative to |
| (50 ng) | | | | mock (±0.07 SEM) |
| 5MOU SDF-1 | 10 | 31.08 ± 0.12 | 1.36 | 2.6-fold relative to |
| (10 ng) | | | | mock (±0.12 SEM) |

As shown in Table 4, direct comparison of WT versus 5MOU-modified SDF-1 mRNA at each matched dose revealed statistically significant reductions in IL-6 expression at every concentration tested, with all comparisons surviving Bonferroni correction (P<0.0036). Thus, at each concentration tested, the 5MOU modification reduced IL-6 inflammatory induction by approximately 4-fold to 172-fold compared to the corresponding wild-type SDF-1 mRNA.

TABLE 4
IL-6 Fold Change vs. Mock Control (±SEM)
and WT vs. 5MOU Statistical Comparisons

| | | | Fold- | | |
|---|---|---|---|---|---|
| Dose | WT IL-6 | 5MOU IL-6 | Reduction | P-value | |
| (ng/ | Fold vs | Fold vs | (WT ÷ | (WT vs | Signifi- |
| well) | Mock | Mock | 5MOU) | 5MOU) | cance |
| 10 | 10.0 ± 0.7 | 2.6 ± 0.1 | 3.9× | 0.00007 | *** |
| 50 | 56.5 ± 11.0 | 1.7 ± 0.1 | 32.5× | 0.00006 | *** |
| 100 | 144.8 ± 9.9 | 2.0 ± 0.6 | 71.5× | 0.00013 | *** |
| 250 | 709.5 ± 60.3 | 4.1 ± 0.3 | 172.2× | <0.0001 | *** |
| 500 | 785.7 ± 150.3 | 15.6 ± 3.2 | 50.2× | 0.00016 | *** |

n = 3 per condition.
*** P < 0.0036 (Bonferroni-corrected threshold for 14 comparisons).
Fold-reduction = (WT fold change) ÷ (5MOU fold change).

As shown in Table 5 below, vehicle control (Lipofectamine MessengerMAX™ only) did not itself contribute meaningfully to the observed inflammatory response. The LPS positive control confirmed that the assay system was capable of detecting NF-κB-mediated inflammatory activation. Importantly, the response to wild-type (unmodified) GFP mRNA at 500 ng/well demonstrated that the inflammatory response to unmodified mRNA is not specific to the SDF-1 sequence but rather attributable to the presence of unmodified uridine in exogenous mRNA which supports 5MOU modification being responsible for the observed attenuation of innate immune activation.

TABLE 5
Control Conditions - IL-6 Fold Change vs. Mock

| | IL-6 Fold | P-value | Signifi- |
|---|---|---|---|
| Control Condition | vs Mock | (vs Mock) | cance |
| Mock (untreated) | 1.00 (reference) | — | — |
| Vehicle (LipoMMAX only) | 2.02 | 0.145 | ns |
| LPS (positive control) | 48.0 | 0.00002 | *** |
| WT GFP mRNA (500 ng) | 11.3 ± 2.5 | 0.00056 | *** |

ns = not significant.
*** P < 0.0036 (Bonferroni-corrected).

These results demonstrate an unexpected and advantageous property of the nucleoside-modified compositions disclosed herein: in addition to enhanced mRNA stability and dramatically increased SDF-1 protein expression (as disclosed in FIGS. 1 and 2), the complete substitution of uridine with 5-Methoxyuridine in SEQ ID NO.: 1 confers substantial attenuation of the innate immune/inflammatory response that is otherwise induced by the corresponding wild-type SDF-1 mRNA (SEQ ID NO.: 3) in human cells. At therapeutically relevant concentrations (10-100 ng/well), the 5MOU-modified SDF-1 mRNA maintained IL-6 levels near baseline (approximately 1.7- to 2.6-fold vs. mock), while the corresponding wild-type mRNA induced 10- to 145-fold IL-6 upregulation. Even at the highest concentration tested (500 ng/well), the 5MOU modification reduced IL-6 induction by approximately 50-fold relative to wild-type (15.6-fold vs. 785.7-fold).

Inflammation in penile tissues contributes to tissue fibrosis, smooth muscle apoptosis, endothelial dysfunction, and impaired nitric oxide signaling-all of which are pathological hallmarks of erectile dysfunction. The 5MOU-modified SDF-1 mRNA compositions disclosed herein (SEQ ID NO.: 1) advantageously achieve high-level SDF-1 protein expression (as shown in FIGS. 1 and 2) while simultaneously substantially reducing the induction of detrimental inflammatory responses (as shown in FIG. 4), thereby providing a composition that is uniquely suited for the treatment of erectile dysfunction in penile tissues.

The foregoing description provides embodiments of the invention by way of example only. It is envisioned that other embodiments may perform similar functions and/or achieve similar results. Any and all such equivalent embodiments and examples are within the scope of the present invention and are intended to be covered by the appended claims.

What is claimed is:

1. A composition comprising a nucleotide sequence as set forth in SEQ ID NO: 1 and/or having 95% homology to SEQ ID NO:1, more preferably having 99% homology to SEQ ID NO: 1 wherein each uracil loci in SEQ ID NO: 1 has been substituted with 5-Methoxyuridine (5 MOU) and/or N1-Methylpseudouridine (N1).

2. The composition of claim 1, wherein each uracil loci in SEQ ID NO: 1 has been substituted with 5-Methoxyuridine (5 MOU).

3. The composition of claim 1, wherein each uracil loci in SEQ ID NO: 1 has been substituted with N1-Methylpseudouridine (N1).

4. The composition of claim 1, further comprising a carrier, the carrier having SEQ ID NO: 1 therein and/or thereon and/or wherein the carrier is conjugated to SEQ ID NO: 1.

5. The composition of claim 4, wherein the carrier is a polymeric based carrier, preferably polyethylene glycol-poly(disulfide amine) (PEG-PDSA); or wherein the carrier is a lipid nanoparticle based carrier.

6. A polypeptide expressed from and/or corresponding to a nucleotide sequence as set forth in SEQ ID NO: 1 and/or having 95% homology to SEQ ID NO: 1, more preferably having 99% homology to SEQ ID NO: 1.

7. A method of treating and/or preventing erectile dysfunction in a subject in need thereof, the method comprising:
(a) contacting human penile cells and/or human penile tissue(s) with a composition having a nucleotide sequence as set forth in SEQ ID NO: 1 and/or having 95% homology to SEQ ID NO: 1, more preferably having 99% homology to SEQ ID NO: 1 wherein each uracil loci in SEQ ID NO: 1 has been substituted with 5-Methoxyuridine (5 MOU) and/or N1-Methylpseudouridine (N1).

8. The method of claim 7, further comprising:
(b) expressing in the human penile cells and/or human penile tissue(s) a polypeptide from and/or corresponding to the nucleotide sequence as set forth in SEQ ID NO: 1 and/or having 95% homology to SEQ ID NO:1, more preferably having 99% homology to SEQ ID NO: 1.

9. The method of claim 7, wherein the human cells and/or human penile tissue(s) are comprised of human fibroblasts and more preferably human penile fibroblasts, human endothelial cells and more preferably human penile endothelial cells, or a combination thereof.

10. The method of claim 7, wherein the composition of step (a) further comprises a carrier, the carrier having SEQ ID NO: 1 therein and/or thereon and/or wherein the carrier is conjugated to SEQ ID NO: 1.

11. The method of claim 7, wherein the carrier is a polymeric based carrier, preferably polyethylene glycol-poly(disulfide amine) (PEG-PDSA); or wherein the carrier is a lipid nanoparticle based carrier.

12. The method of claim 7, wherein each uracil loci in SEQ ID NO: 1 has been substituted with 5-Methoxyuridine (5 MOU).

13. The method of claim 8, wherein the expression of the polypeptide within the human penile tissues and/or human cells and/or the human penile cells is 100-fold to 5000-fold higher than expression of wild-type Stromal Derived Factor-1 (SDF-1) (SEQ ID NO: 3) in human penile tissues and/or human cells and/or human penile cells contacted with a same concentration of SEQ ID NO: 3 as the concentration of SEQ ID NO: 1 in step (a).

14. The method of claim 8, wherein expression of at least one pro-inflammatory cytokine within the human penile tissues and/or human cells and/or the human penile cells is 4-fold to 172-fold lower than expression of the at least one pro-inflammatory cytokine in human penile tissues and/or human cells and/or human penile cells contacted with a same concentration of SEQ ID NO: 3 as the concentration of SEQ ID NO: 1.


SEQUENCE LISTING (5 sequences)

SEQ ID NO: 1 - RNA, 270 nt, synthetic construct
  source 1..270: mol_type = other RNA
atgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagtaa

SEQ ID NO: 2 - DNA, 270 bp, synthetic construct
  source 1..270: mol_type = other DNA
atgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagtaa

SEQ ID NO: 3 - RNA, 270 nt, synthetic construct; identical to SEQ ID NO: 1
  source 1..270: mol_type = other RNA

SEQ ID NO: 4 - DNA, 47 bp, synthetic construct
  source 1..47: mol_type = other DNA
aggaaataagagagaaaagaagagtaagaagaaatataagagccacc

SEQ ID NO: 5 - DNA, 93 bp, synthetic construct
  source 1..93: mol_type = other DNA
gctgccttctgcggggcttgccttctggccatgcccttcttctctcccttgcacctgtacctcttggtctttgaataaagcctgagtaggaag